(19) United States
(12) Patent Application Publication
(10) Pub. No.: US 2018/0143153 A1
(43) Pub. Date: May 24, 2018

(54) BIO-CELL DETECTION APPARATUS AND BIO DETECTION METHOD

(71) Applicant: RICHTEK TECHNOLOGY CORPORATION, Chupei City (TW)

(72) Inventors: Chao-Shiun Wang, Chupei City (TW); Chang-Yu Ho, Chupei City (TW)

(21) Appl. No.: 15/724,076

(22) Filed: Oct. 3, 2017

Related U.S. Application Data
(60) Provisional application No. 62/426,151, filed on Nov. 23, 2016.

(30) Foreign Application Priority Data
Aug. 10, 2017 (CN) 201710680777.0

(51) Int. Cl. G01N 27/22; C12Q 1/68

(52) U.S. Cl. CPC G01N 27/228; C12Q 1/6825

(57) ABSTRACT

The present invention provides a bio-cell detection apparatus and a bio detection method. The bio detection method includes: in absence of a biological reagent being introduced, pre-calibrating an initial value of a bio-cell detection chip. In absence of a sample to be tested in the bio-cell detection chip, introducing only the biological reagent into the bio-cell detection chip; and, measuring a first electrical parameter between a pair of opposing electrodes of the bio-cell detection chip. Introducing both the biological reagent and the sample to be tested into the bio-cell detection chip at the same time; and, measuring a second electrical parameter between the pair of opposing electrodes of the bio-cell detection chip. Comparing the second electrical parameter and the first electrical parameter, to determine whether a target biomolecule to be detected is present.

### CROSS REFERENCE

The present invention claims priorities to CN 201710680777.0, filed on Aug. 10, 2017, and U.S. provisional application 62/426,151, filed on Nov. 23, 2016.

### BACKGROUND OF THE INVENTION

### Field of Invention

The present invention relates to a bio-cell detection apparatus and a bio detection method; particularly, it relates to such bio-cell detection apparatus and bio detection method having adaptive calibration mechanism, which are applicable to testing different biological targets.

### Description of Related Art

Please refer to FIG. 1, which shows a flowchart of a conventional bio detection method. As shown in FIG. 1, in this conventional bio detection method, first, a distilled water is introduced into a conventional bio-cell detection chip, to measure an electrical parameter (such as an impedance value or a capacitance value) between a pair of opposing electrodes of the conventional bio-cell detection chip (referring to step S11 in FIG. 1). Next, referring to step S12 in FIG. 1, the distilled water is removed (the above steps are performed before a sample to be tested is added and a biological reaction takes place, in order to confirm an initial value).

Next, referring to step S13 in FIG. 1, a biological reagent and a sample to be tested are introduced into the bio-cell detection chip (this step is the step whereby a biological reaction takes place).

Subsequently, referring to step S14a in FIG. 1, the biological reagent introduced into the bio-cell detection chip in step S13 is removed. After that, in step S14b, distilled water is again introduced into the bio-cell detection chip, so that, subsequently in step S14c, the electrical parameter (the impedance value or the capacitance value) between the electrodes of the bio-cell detection chip can be measured again.

Lastly, the electrical parameter (the impedance value or the capacitance value) of the step S14c is compared to the electrical parameter (the impedance value or the capacitance value) of the step S11, that is, the initial value before and the biological reaction and the value measured after the biological reaction are compared with each other, to obtain a test result, such as to determine whether or not a target biomolecule is present.

The drawbacks of such conventional bio detection method are: (1) It requires adding and removing distilled water, causing the process complicated. (2) The step of removing the biological reagent in step S14a will also remove a portion of the reactant, which will undesirably affect the readout of a final measurement and render the measurement inaccurate.

For relevant details related to a bio-cell detection apparatus and a bio detection method, one can refer to the followings: U.S. Patent Publication No. 2004/0110277 and U.S. Patent Publication No. 2013/0143775.

In view of the above, to overcome the drawbacks in the prior art, the present invention propose a bio-cell detection apparatus and a bio detection method having adaptive calibration mechanism, which are applicable to testing different biological targets.

### SUMMARY OF THE INVENTION

From one perspective, the present invention provides a bio detection method, comprising the steps of: (A) pre-calibrating an initial value of a bio-cell detection chip in absence of a biological reagent in the bio-cell detection chip; (B) introducing the biological reagent into the bio-cell detection chip in absence of a sample to be tested in the bio-cell detection chip, and measuring a first electrical parameter between a pair of opposing electrodes of the bio-cell detection chip; (C) introducing the biological reagent and the sample to be tested into the bio-cell detection chip, and measuring a second electrical parameter between the pair of opposing electrodes of the bio-cell detection chip; and (D) comparing the second electrical parameter of the step (C) and the first electrical parameter of the step (B), to determine whether or not a target biomolecule to be detected is present.

In one embodiment, the step (A) includes: (A1) outputting the initial value of the bio-cell detection chip; (A2) determining whether the initial value falls within an acceptable range; (A3) when the initial value falls within the acceptable range, proceeding to the step (B); and (A4) when the initial value does not fall within the acceptable range, calibrating the initial value so that the initial value falls within the acceptable range.

In one embodiment, the step (A) further includes: inputting an excitation signal to the bio-cell detection chip.

In one embodiment, the step (B) includes: (B1) outputting a first output signal which is related to the first electrical parameter; (B2) determining whether the first output signal reaches a basic level; (B3) when the first output signal reaches the basic level, proceeding to the step (C); and (B4) when the first output signal does not reach the basic level, adjusting a level of the first output signal to the basic level.

In one embodiment, the step (B) further includes: inputting an excitation signal to the bio-cell detection chip.

In one embodiment, the step (C) includes: (C1) outputting a second output signal which is related to the second electrical parameter.

In one embodiment, the step (C) further includes: inputting an excitation signal to the bio-cell detection chip.

In one embodiment, the biological reagent includes a buffer solution or an electrolyte solution.

In one embodiment, the first electrical parameter includes an impedance value or a capacitance value and the second electrical parameter respectively includes an impedance value or a capacitance value.

From another perspective, the present invention provides a bio-cell detection apparatus, comprising: at least one bio-cell detection chip, configured to operably carry a biological reagent and/or a sample to be tested; a detection circuit, configured to operably output an initial value and/or a measure signal of the bio-cell detection chip, wherein the measure signal includes: a first electrical parameter measured between a pair of opposing electrodes of the bio-cell detection chip when the biological reagent is introduced into the bio-cell detection chip but the sample to be tested is not introduced into the bio-cell detection chip; and/or a second electrical parameter measured between the pair of opposing electrodes of the bio-cell detection chip when both the biological reagent and the sample to be tested are introduced into the bio-cell detection chip at the same time; a control circuit, configured to operably determine whether the initial value falls within an acceptable range, or determine whether the first electrical parameter reaches a basic level; a calibration circuit, configured to operably pre-calibrate the initial value within the acceptable range according to a determination result outputted from the control circuit when the biological reagent is not introduced into the bio-cell detection chip; and an adjustment circuit, configured to operably adjust a level of the first output signal to the basic level according to the determination result outputted from the control circuit.

In one embodiment, the bio-cell detection apparatus further includes a multiplexer, which is configured to operably multiplex control a plurality of bio-cell detection chips, wherein the plurality of bio-cell detection chips are arranged in a 1×N array.

In one embodiment, the bio-cell detection apparatus further includes: at least two multiplexers, which are configured to operably multiplex control a plurality of bio-cell detection chips, wherein the plurality of bio-cell detection chips are arranged in a N×N array.

In one embodiment, there are plural bio-cell detection chips and the plural bio-cell detection chips are arranged in serial.

In one embodiment, there are plural bio-cell detection chips and the plural bio-cell detection chips are arranged in parallel.

In one embodiment, the biological reagent includes a buffer solution or an electrolyte solution.

In one embodiment, the first electrical parameter includes an impedance value or a capacitance value and the second electrical parameter respectively includes an impedance value or a capacitance value.

From yet another perspective, the present invention provides a bio detection method, comprising the steps of: (A) pre-calibrating a first initial value of a reference bio-cell detection chip in absence of a biological reagent in the reference bio-cell detection chip; (B) introducing the biological reagent into the reference bio-cell detection chip in absence of a sample to be tested in the reference bio-cell detection chip, and measuring a first electrical parameter between a pair of opposing electrodes of the reference bio-cell detection chip; (C) pre-calibrating a second initial value of a test bio-cell detection chip in absence of the biological reagent in the test bio-cell detection chip; (D) introducing the biological reagent and the sample to be tested into the bio-cell detection chip, and measuring a second electrical parameter between a pair of opposing electrodes of the test bio-cell detection chip; and (E) comparing the second electrical parameter of the step (D) and the first electrical parameter of the step (B), to determine whether or not a target biomolecule to be detected is present.

In one embodiment, the biological reagent includes a buffer solution or an electrolyte solution.

In one embodiment, the first electrical parameter includes an impedance value or a capacitance value and the second electrical parameter respectively includes an impedance value or a capacitance value.

From still another perspective, the present invention provides a bio-cell detection apparatus, comprising: a reference bio-cell detection chip, configured to operably carry a biological reagent; at least one test bio-cell detection chip, configured to operably carry the biological reagent and/or a sample to be tested; a detection circuit, configured to operably output a first initial value of the reference bio-cell detection chip and/or a second initial value of the test bio-cell detection chip, and configured to operably compare a first measure signal of the reference bio-cell detection chip and a second measure signal of the test bio-cell detection chip; wherein the first measure signal includes: a first electrical parameter, measured between a pair of opposing electrodes of the reference bio-cell detection chip when the biological reagent is introduced into the reference bio-cell detection chip but the sample to be tested is not introduced into the reference bio-cell detection chip; wherein the second measure signal includes: a second electrical parameter measured between a pair of opposing electrodes of the test bio-cell detection chip when the biological reagent and the sample to be tested are introduced into the test bio-cell detection chip; a control circuit, configured to operably determine whether the first initial value of the reference bio-cell detection chip or the second initial value of the test bio-cell detection chip falls within an acceptable range and/or determine whether the first electrical parameter reaches a basic level; a calibration circuit, configured to operably pre-calibrate the first initial value within the acceptable range according to a determination result outputted from the control circuit when the biological reagent is not introduced into the reference bio-cell detection chip, and/or to operably pre-calibrate the second initial value within the acceptable range according to the determination result outputted from the control circuit when the biological reagent is not introduced into the test bio-cell detection chip; and an adjustment circuit, configured to operably adjust a level of the first output signal to the basic level according to the determination result outputted from the control circuit.

In one embodiment, the bio-cell detection apparatus further includes: a multiplexer, which is configured to operably multiplex control the reference bio-cell detection chip and a plurality of test bio-cell detection chips, wherein the reference bio-cell detection chip and the plurality of test bio-cell detection chips are arranged in a 1×N array.

In one embodiment, the bio-cell detection apparatus further includes: at least two multiplexers, which are configured to operably multiplex control the reference bio-cell detection chip and a plurality of test bio-cell detection chips, wherein the reference bio-cell detection chip and the plurality of test bio-cell detection chips are arranged in a N×N array.

The objectives, technical details, features, and effects of the present invention will be better understood with regard to the detailed description of the embodiments below, with reference to the attached drawings.

### BRIEF DESCRIPTION OF THE DRAWINGS

FIG. 1 shows a flowchart of a conventional bio detection method.

FIG. 2 shows a flowchart of a bio detection method according to a first embodiment of the present invention.

FIG. 3 shows a more specific embodiment of the flowchart of FIG. 2.

FIG. 4 shows, in correspondence with FIGS. 2˜3, a schematic block diagram of a bio-cell detection apparatus according to a first embodiment of the present invention.

FIG. 5 shows a flowchart of a bio detection method according to a second embodiment of the present invention.

FIG. 6 shows, in correspondence with FIG. 5, a schematic block diagram of a bio-cell detection apparatus according to a second embodiment of the present invention.

FIG. 7 shows a schematic block diagram of a bio-cell detection apparatus according to a third embodiment of the present invention.

FIG. 8 shows a schematic block diagram of a bio-cell detection apparatus according to a fourth embodiment of the present invention.

FIG. 9 shows a schematic block diagram of a bio-cell detection apparatus according to a fifth embodiment of the present invention.

FIG. 10 shows a schematic block diagram of a bio-cell detection apparatus according to a sixth embodiment of the present invention.

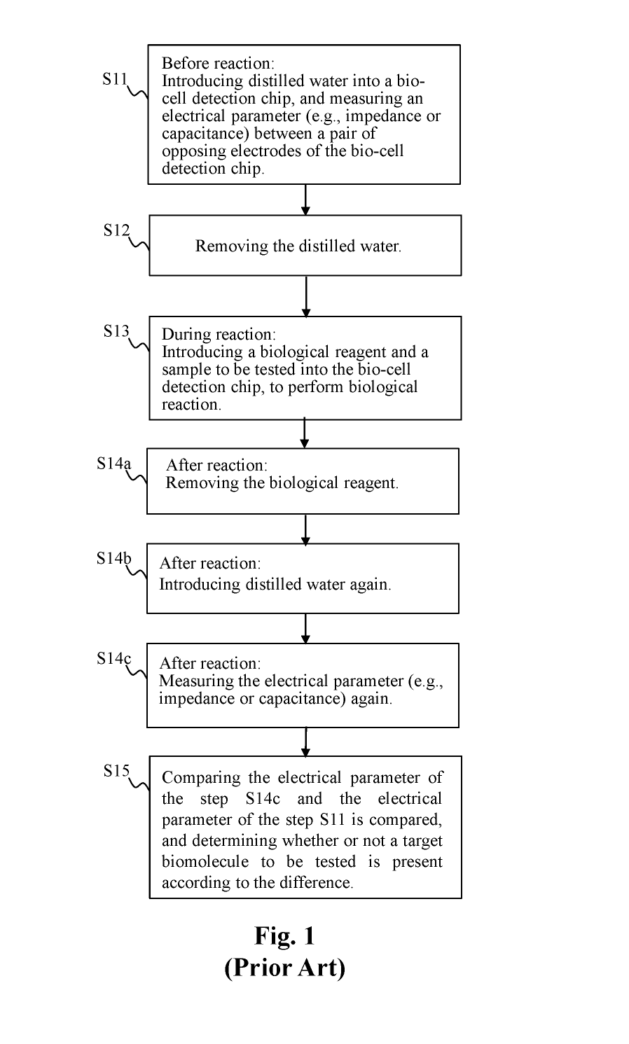

### DESCRIPTION OF THE PREFERRED EMBODIMENTS

The above and other technical details, features and effects of the present invention will be will be better understood with regard to the detailed description of the embodiments below, with reference to the drawings. The drawings as referred to throughout the description of the present invention are for illustration only, to show the interrelations between the regions and the process steps, but not drawn according to actual scale.

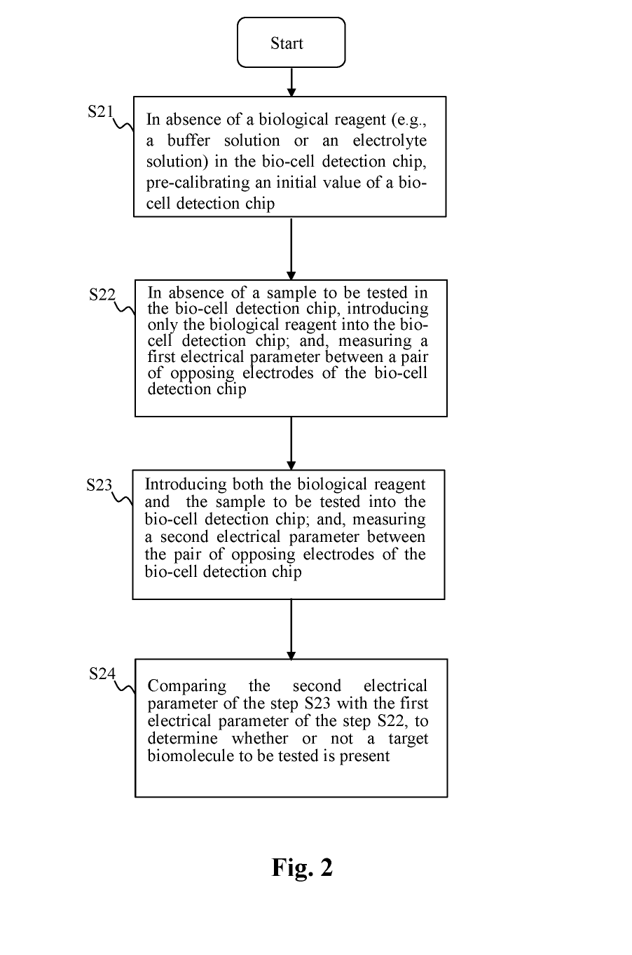

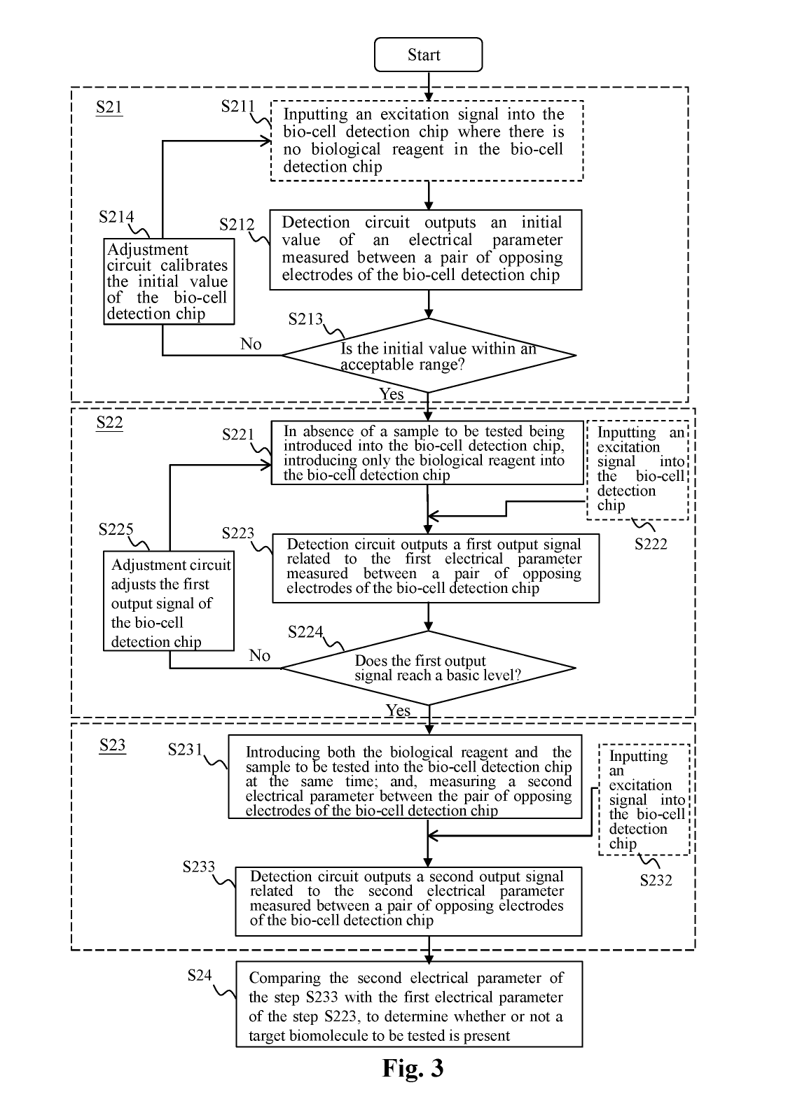

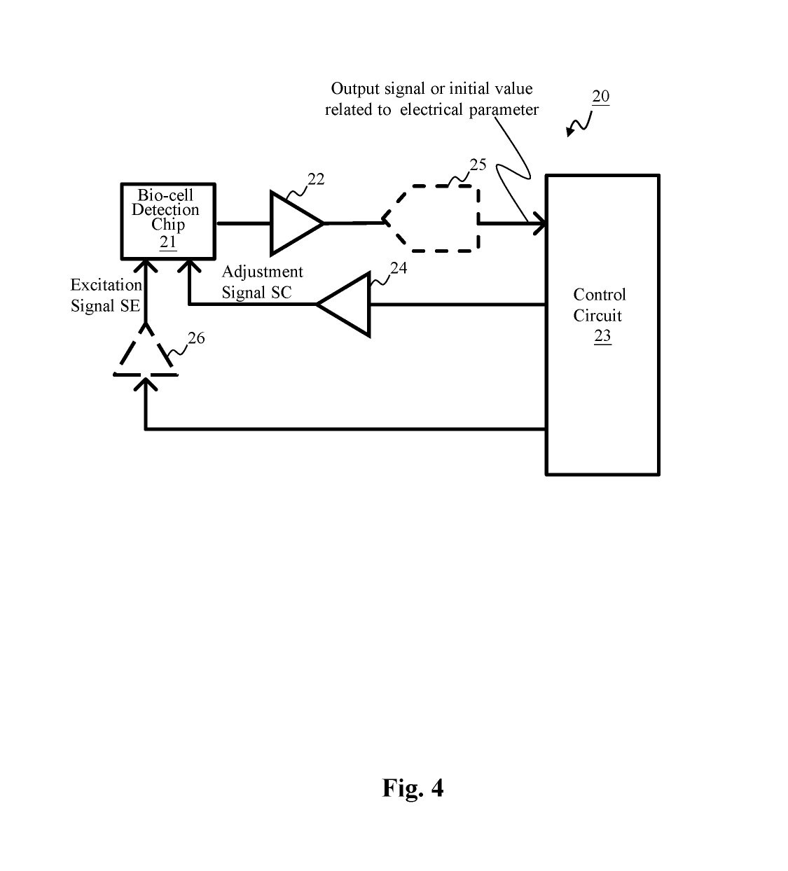

Please refer to FIGS. 2˜3 in conjugation with FIG. 4. FIG. 2 shows a flowchart of a bio detection method according to a first embodiment of the present invention. FIG. 3 shows a more specific embodiment of the flowchart of FIG. 2. FIG. 4 shows, in conjugation with FIGS. 2˜3, a schematic block diagram of a bio-cell detection apparatus according to a first embodiment of the present invention.

As shown in FIG. 4, a bio-cell detection apparatus 20 comprises: a bio-cell detection chip 21, a detection circuit 22, a control circuit 23, a calibration circuit 24 and an adjustment circuit 26. In addition, the bio-cell detection apparatus 20 of this embodiment can optionally comprise an analog-to-digital conversion circuit (ADC) 25. In this embodiment, the bio-cell detection apparatus 20 includes one bio-cell detection chip 21 as an example, but this is for illustrative purpose, not for limiting the scope of the present invention. In other embodiments, there may be plural bio-cell detection chip 21, not limited to only one bio-cell detection chip 21. In one embodiment, the bio-cell detection chip 21 can include a recess or hollow structure for carrying a biological reagent and/or a sample to be tested.

This embodiment is different from the prior art in that: as shown in FIG. 2, in absence of a biological reagent (which can be, for example but not limited to, a buffer solution or an electrolyte solution, depending on the purpose of the bio detection), an initial value of the bio-cell detection chip 21 is pre-calibrated in advance (referring to step S21 in FIG. 2 and step S214 in FIG. 3). Please refer to FIG. 3, which shows how the bio-cell detection apparatus 20 pre-calibrates the initial value of the bio-cell detection chip 21. As shown in FIG. 3, in one embodiment, the bio-cell detection apparatus 20 inputs an excitation signal SE to the bio-cell detection chip 21 via the adjustment circuit 26 (referring to step S211 in FIG. 3). Next, the initial value of the bio-cell detection chip 21 is outputted through the detection circuit 22 (referring to step S212 in FIG. 3). The term “initial value” as described herein is meant to indicate, for example but not limited to, an electrical parameter measured between a pair of opposing electrodes of the bio-cell detection chip 21; such electrical parameter can be, for example but not limited to, an impedance value or a capacitance value.

In biological detection, a biomolecule will react with a given reagent, to cause a change of a capacitance value between the electrodes. The capacitance change is affected by two factors: one is a capacitance (Cbio) on the surface of the sensing electrode after the biomolecules is hybridized with the receptor attached on the sensing electrode, while the other is a capacitance (Cgap) between opposing electrodes. The total capacitance change can be represented by the following equation:

$${C}_{Tot} = \frac{1}{\frac{1}{{C}_{bio}} + \frac{1}{{C}_{gap}}}$$

$${C}_{gap} = \frac{Q}{V} = ɛ \frac{A}{t}$$

wherein, CTot denotes a total change of the capacitance value, A denotes an area of the electrode, t denotes a distance between the electrodes, ε denotes a dielectric constant of the buffer solution or the electrolyte solution between electrodes. Because the reaction between the biomolecule and the reagent will affect not only the capacitance value but also the impedance value of the bio-cell detection chip 21, the detection can also be performed by measuring the impedance value of the bio-cell detection chip 21.

The excitation signal SE shown in step S211 of FIG. 3 is not necessarily required to be inputted to the bio-cell detection chip 21. This step S211 is an optional step (and this is why step S211 in FIG. 3 is illustrated by a dotted rectangle). In other words, for certain detections, the initial value of the bio-cell detection chip 21 can be directly measured without providing an excitation signal SE to the bio-cell detection chip 21. On the other hand, in this embodiment, preferably, it can be designed that the excitation signal SE can be adaptively adjusted whenever necessary. That is, the excitation signal SE can be adjusted according to different characteristics of different samples to be tested, so that the initial value of the bio-cell detection chip 21 can be adaptively set within an acceptable range. For example, for certain types of samples to be tested, during a biological reaction, a change of the capacitance value might fall within an order of magnitude, whereas, for other types of samples to be tested, during a biological reaction, a change of the capacitance value might fall with another order of magnitude; the two orders of magnitude may have a hundred-fold difference. Or, for another example, for certain types of samples to be tested, during a biological reaction, the adoption of a buffer solution or an electrolyte solution used in the biological reaction might induce a more sensitive capacitance change, whereas, for other types of samples to be tested, during a biological reaction, the adoption of a buffer solution or an electrolyte solution used in the biological reaction might induce a less sensitive capacitance change, and the difference may be huge. Accordingly, through adjusting the excitation signal SE, the initial value can be set to fall within an acceptable range, and the bio-cell detection apparatus 20 of the present invention can become a multiple-use bio-cell detection apparatus 20, which is not limited to detecting only single type of target biomolecule.

For example, referring to step S213 in FIG. 3, in this embodiment, preferably, the bio-cell detection apparatus 20 can determine whether the initial value in step S212 falls within an acceptable range. When this initial value falls within the acceptable range, the bio-cell detection apparatus 20 can subsequently proceed to step S22 in FIGS. 2˜3 (the details will be described later). When this initial value does not fall within the acceptable range, the calibration circuit 24 (referring to FIG. 4) outputs a calibration signal SC to adjust the initial value to fall within the acceptable range (referring to step S214 in FIG. 3). Thus, this embodiment can ensure that the initial value of the bio-cell detection chip falls within an accurate order of magnitude.

In one embodiment, the calibration circuit 24 can calibrate the initial value to fall within the acceptable range by adjusting a current, a voltage, a resistance or a capacitance.

It is noteworthy that the prior art does not possess the above-mentioned feature and advantage; the prior art cannot adaptively calibrate the initial value of the bio-cell detection chip 21, and therefore the conventional bio-cell detection apparatus is limited to detecting only single type of target biomolecule, unable for multiple-use.

Next, referring to step S22 in FIGS. 2˜3, in absence of a sample to be tested (e.g., within which a target biomolecule such as a DNA molecule for hybridization reaction is to be detected) in the bio-cell detection chip 21, introducing only the biological reagent (e.g., a buffer solution or an electrolyte solution) into the bio-cell detection chip 21. And, in step S22 in FIGS. 2˜3, a first electrical parameter between a pair of opposing electrodes of the bio-cell detection chip 21 is measured. As described above, this first electrical parameter can be a capacitance value or an impedance value (referring to step S22 in FIGS. 2˜3).

Next, as shown in FIG. 3, the detection circuit 22 is configured to operably output a first output signal which is related to the first electrical parameter of the bio-cell detection chip 21 (the first output signal indicates a detection result when only the biological reagent is introduced into the bio-cell detection chip 21 in absence of a sample to be tested in the bio-cell detection chip 21). The step S222 is similar to step S211, namely, in step S222, the bio-cell detection apparatus 20 can also input or adjust the excitation signal SE via the adjustment circuit 26 (referring to step S222 in FIG. 3), so that an appropriate order of magnitude can be achieved. As described above, this step S222 is optional (and this is why step S222 in FIG. 3 is illustrated by a dotted rectangle).

The step S22 is also a feature of the present invention which is different from the prior art. As shown in FIG. 3, the bio-cell detection apparatus 20 can, in step S224 in FIG. 3, determine whether the first output signal (indicating when only the biological reagent is introduced into the bio-cell detection chip 21 in absence of a sample to be tested in the bio-cell detection chip 21) of step S223 reaches a basic level which can be easily detected and identified. When the first output signal of step S223 reaches the basic level, this embodiment will subsequently proceed to the step S23 (the details will be described later). When the first output signal of step S223 does not reach the basic level, in this embodiment, preferably, through the excitation signal SE outputted from the adjustment circuit 26, a level of the first output signal can be adjusted to reach the basic level, so that the first output signal can be detectable.

In one embodiment, the adjustment circuit 26 can automatically adjust the feedback signal according to different electrical characteristics of different target biomolecules via, for example but not limited to, a DC current, a voltage or an alternating period and amplitude of AC power, to achieve an optimum amplification of the detection circuit 22. As a result, a level of the first output signal is adjusted to reach the basic level, so that the first output signal can be detected.

Next, referring to step S23 in FIGS. 2˜3, both the biological reagent (e.g., a buffer solution or an electrolyte solution) and the sample to be tested (e.g., within which a target biomolecule such as a DNA molecule for hybridization reaction is to be detected) are introduced into the bio-cell detection chip 21. And, a second electrical parameter between the pair of opposing electrodes of the bio-cell detection chip 21 is measured. As described above, this second electrical parameter can be a capacitance value or an impedance value (referring to step S23 in FIG. 2).

Note that, that “introducing the biological reagent is introduced into the bio-cell detection chip 21” in step S231 can be, in one embodiment, keeping the biological reagent which has been the introduced into the bio-cell detection chip 21 in step S221, or, in another embodiment, introducing new or more biological reagent into the bio-cell detection chip 21. The latter, for example, can be applied to the case wherein the steps S221-S225 consume the biological reagent to a significant amount. Or, in another embodiment, the biological reagent which has been introduced in step S221 can be removed first, and subsequently new biological reagent are re-introduced. It is noteworthy that, during removal of the previously-introduced biological reagent, the sample to be tested will not be taken away together with the removal of the biological reagent, so the readout of the measurement will not be affected.

Next, please refer to FIG. 3. As shown in FIG. 3, the detection circuit 22 is configured to operably output a second output signal which is related to the second electrical parameter of the bio-cell detection chip 21 (the second output signal indicates a detection result when both the biological reagent and the sample to be tested are introduced into the bio-cell detection chip 21) (referring to step S233 in FIG. 3). Optionally, in step S23, the bio-cell detection apparatus 20 can also input an excitation signal SE via the adjustment circuit 26 (referring to step S232 in FIG. 3), which is optional and therefore is illustrated by a dotted rectangle.

Next, as shown in FIG. 3, the second electrical parameter of the step S23 is compared with the first electrical parameter of the step S22, to determine whether or not a target biomolecule to be detected is present. Thus, the bio detection method of this embodiment is completed.

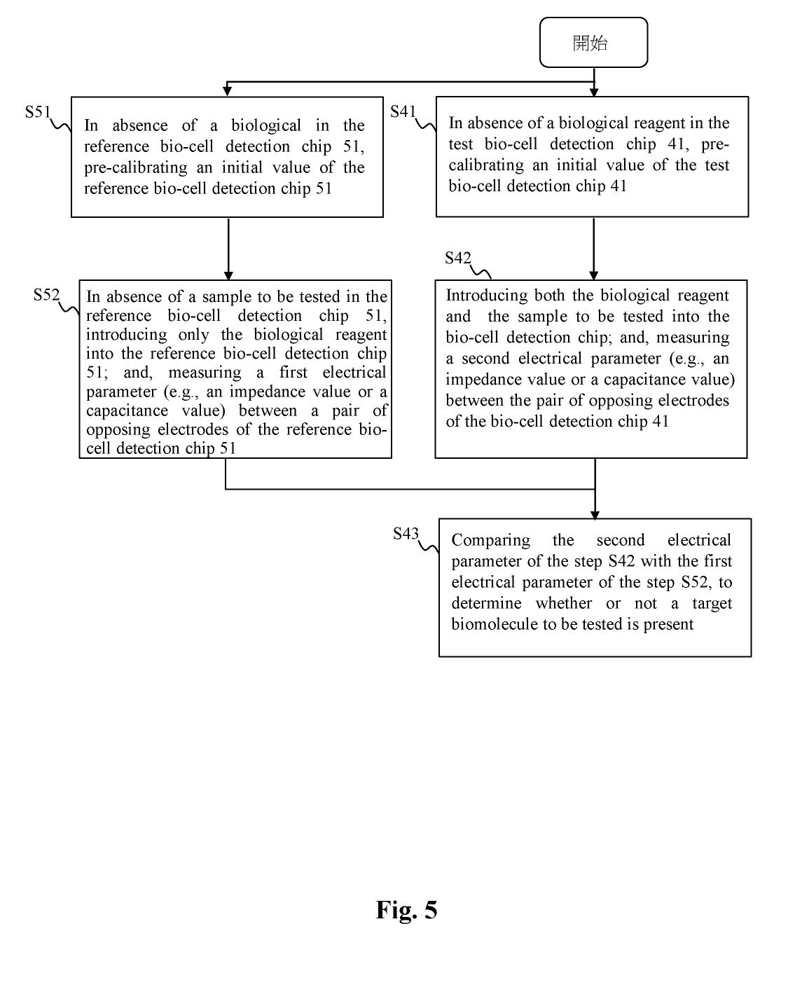

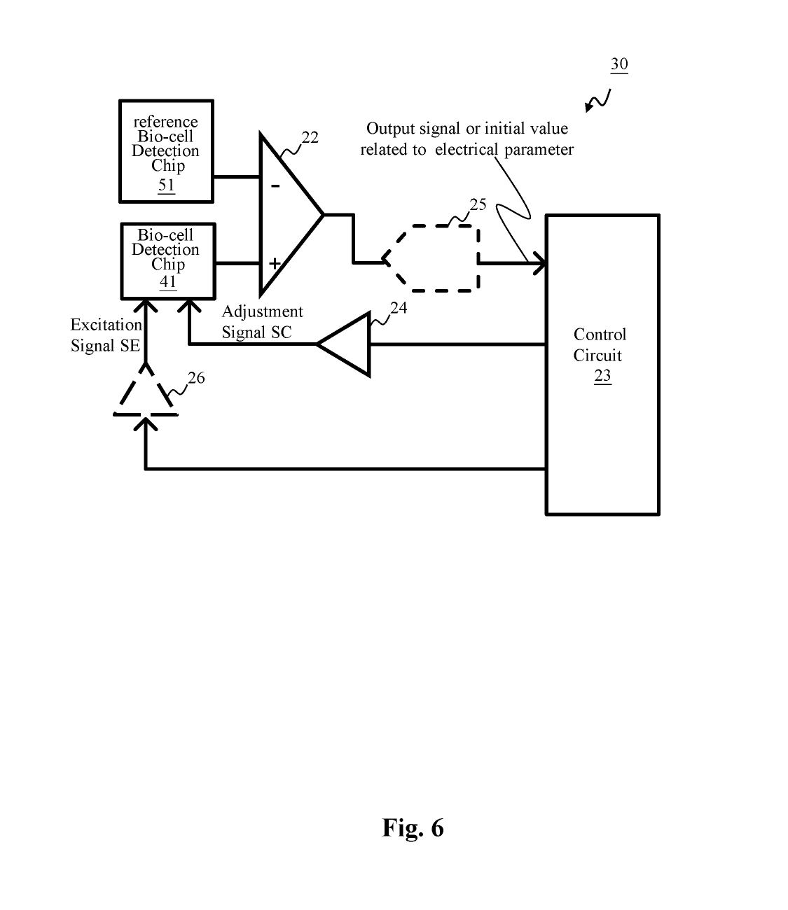

Please refer to FIG. 5 in conjugation with FIG. 6. FIG. 5 shows a flowchart of a bio detection method according to a second embodiment of the present invention. FIG. 6 shows, in conjugation with FIG. 5, a schematic block diagram of a bio-cell detection apparatus according to a second embodiment of the present invention.

As shown in FIG. 6, a bio-cell detection apparatus 30 comprises: a bio-cell detection chip 41, a reference bio-cell detection chip 51, a detection circuit 22, a control circuit 23, a calibration circuit 24 and an adjustment circuit 26. In addition, the bio-cell detection apparatus 30 of this embodiment can optionally comprise an analog-to-digital conversion circuit (ADC) 25. In this embodiment, the bio-cell detection apparatus 30 includes for example one bio-cell detection chip 41, but this is for illustrative purpose, not for limiting the scope of the present invention. In other embodiments, a number of the bio-cell detection chip 41 can be plural and is not limited to only one bio-cell detection chip 41. The bio-cell detection apparatus 30 of this embodiment is different from the bio-cell detection apparatus 20 of the first embodiment in that: the bio-cell detection apparatus 30 of this embodiment includes a reference bio-cell detection chip 51 (the features and the advantages of the reference bio-cell detection chip 51 will be described later).

As shown in FIG. 5, in absence of a biological reagent (which can be, for example but not limited to, a buffer solution or an electrolyte solution, depending on the actual purpose for bio detection) in the reference bio-cell detection chip 51, this embodiment can pre-calibrate an initial value of the reference bio-cell detection chip 51 in advance (referring to step S51 in FIG. 5). Besides, similarly, as shown in FIG. 5, in absence of a biological reagent in the bio-cell detection chip 41, this embodiment can pre-calibrate an initial value of the bio-cell detection chip 41 in advance (referring to step S41 in FIG. 5). Thus, the bio-cell detection apparatus 30 of this embodiment can determine whether the initial value of the step 41 and the initial value of the step 51 fall within an acceptable range. The calibration of the bio-cell detection apparatus 30 of this embodiment is similar to the bio-cell detection apparatus 20, so the details thereof are not redundantly repeated here.

Next, referring to step S52 in FIG. 5, in absence of a sample to be tested (e.g., within which a target biomolecule such as a DNA molecule for hybridization reaction is to be detected) in the reference bio-cell detection chip 51, introducing only the biological reagent (e.g., a buffer solution or an electrolyte solution) into the reference bio-cell detection chip 51. And, step S52 in FIG. 5, a first electrical parameter between a pair of opposing electrodes of the reference bio-cell detection chip 51 is measured. As described above, this first electrical parameter can be a capacitance value or an impedance value (referring to step S52 in FIG. 5). The technique as to how to measure an electrical parameter between a pair of opposing electrodes is well known to those skilled in the art, so the details thereof are not redundantly explained here.

Similarly to the bio-cell detection apparatus 20, the bio-cell detection apparatus 30 of this embodiment can, in step S52 in FIG. 5, determine whether the first output signal which is related to the first electrical parameter (the first output signal indicates a detection result when only the biological reagent is introduced into the reference bio-cell detection chip 51 in absence of a sample to be tested in the reference bio-cell detection chip 51) reaches a basic level. When the first output signal of step S52 does not reach the basic level, in this embodiment, through the excitation signal SE outputted from the adjustment circuit 26, this embodiment can adjust a level of the first output signal to reach the basic level, so that the first output signal becomes detectable. The mechanism to adjust the excitation signal SE outputted from the adjustment circuit 26 can be adjusting the current, voltage, alternating period or alternating amplitude, as described above with reference to the bio-cell detection apparatus 20.

Next, referring to step S42 in FIG. 5, both the biological reagent (e.g., a buffer solution or an electrolyte solution) and the sample to be tested (e.g., within which a target biomolecule such as a DNA molecule for hybridization reaction is to be detected) are introduced into the bio-cell detection chip 41. And, a second electrical parameter between the pair of opposing electrodes of the bio-cell detection chip 41 is measured. As described above, this second electrical parameter can be a capacitance value or an impedance value (referring to step S42 in FIG. 5).

Similarly to the bio-cell detection apparatus 20, the bio-cell detection apparatus 30 of this embodiment can, in step S42 in FIG. 5, determine whether the second output signal which is related to the second electrical parameter (the second output signal indicates a detection result both the biological reagent and the sample to be tested are introduced into the bio-cell detection chip 41) reaches a basic level. When the second output signal of step S52 does not reach the basic level, in this embodiment, through the excitation signal SE outputted from the adjustment circuit 26, this embodiment can also adjust a level of the second output signal to reach the basic level, so that the second output signal becomes detectable. For the sake of proper comparison between the first output signal and the second output signal, the excitation signal SE inputted in step 42 and step 52 should preferably be the same.

Next, as shown in FIG. 5, the second electrical parameter of the step S42 is compared to the first electrical parameter of the step S52, to determine whether or not a target biomolecule to be detected is present. Thus, the bio detection method of this embodiment is completed. The bio-cell detection apparatus 30 of this embodiment is different from the bio-cell detection apparatus 20 of the first embodiment in that: the reference bio-cell detection chip 51 provides a reference (wherein only the biological reagent is introduced into the reference bio-cell detection chip 51 while a sample to be tested is not introduced into the reference bio-cell detection chip 51). Thus, the electrical parameter of the bio-cell detection chip 41 (wherein both the biological reagent and the sample to be tested are introduced into the bio-cell detection chip 41) can be compared with the electrical parameter obtained from the reference bio-cell detection chip 51, to produce a differential measurement result. Accordingly, the detection circuit 22 of the bio-cell detection apparatus 30 is a comparator, which is configured to operably compare the second electrical parameter of step 42 with the first electrical parameter of step 52. The first embodiment can be viewed as a method processed in a serial manner, while, the second embodiment can be viewed as a method processed in a parallel manner.

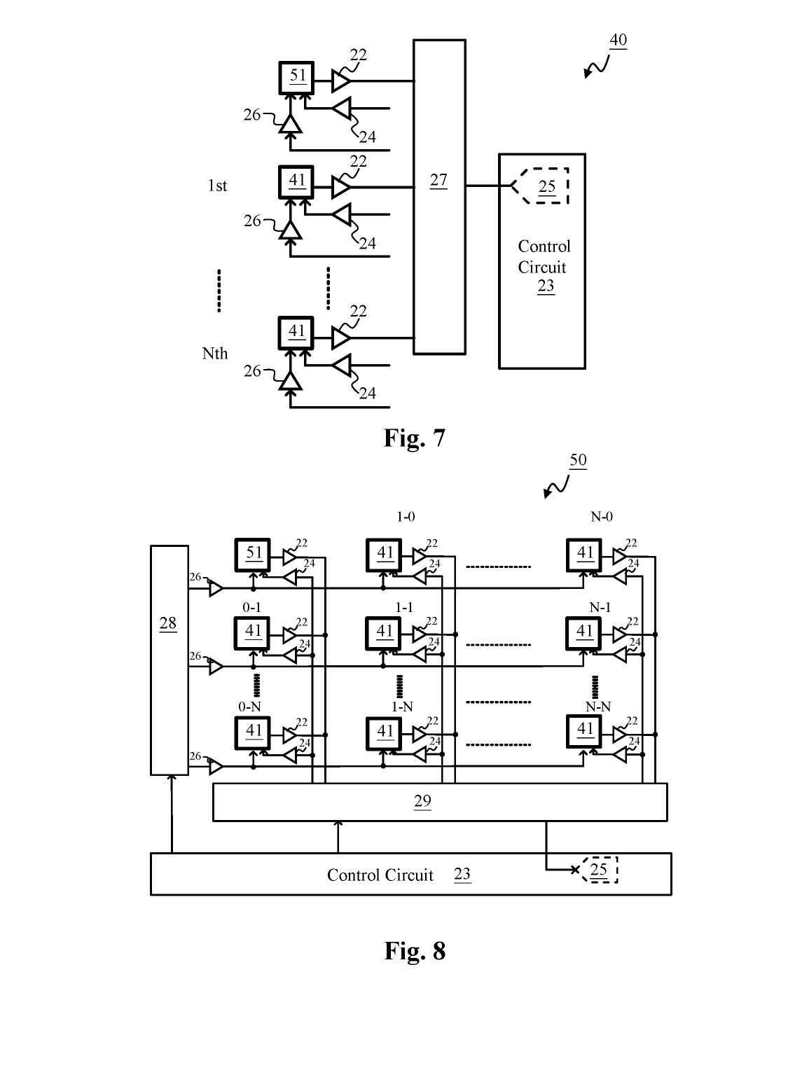

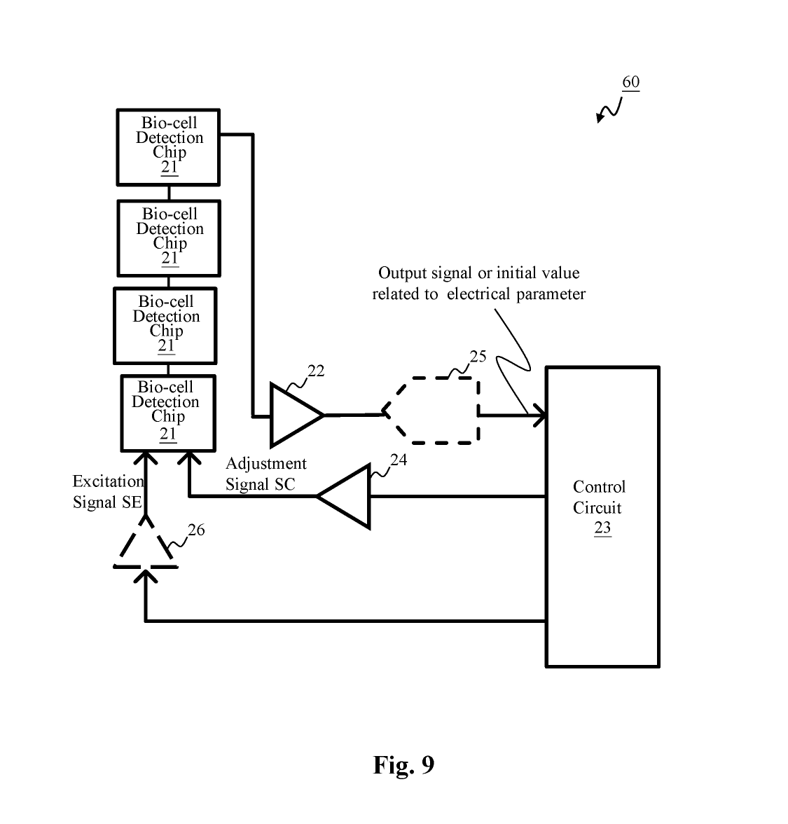

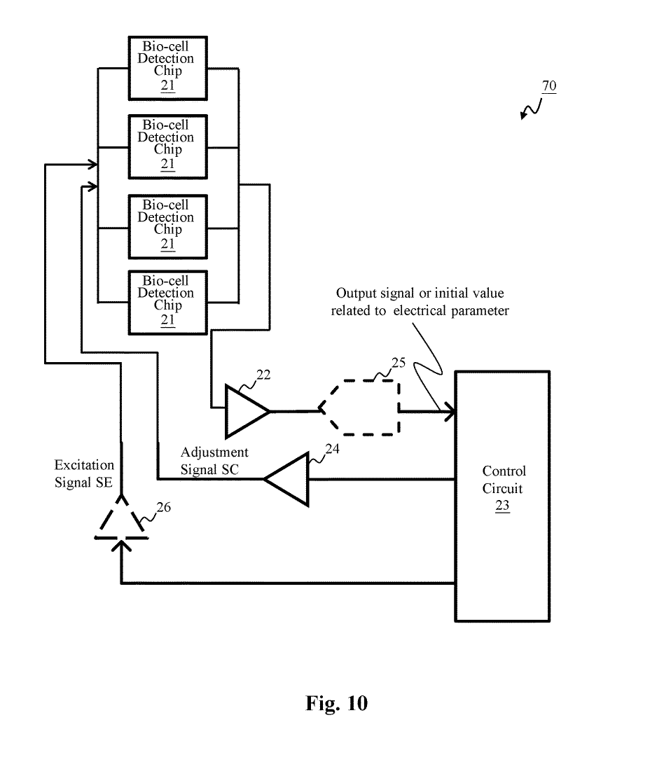

Please refer to FIGS. 7˜10. FIG. 7 shows a schematic block diagram of a bio-cell detection apparatus according to a third embodiment of the present invention. FIG. 8 shows a schematic block diagram of a bio-cell detection apparatus according to a fourth embodiment of the present invention. FIG. 9 shows a schematic block diagram of a bio-cell detection apparatus according to a fifth embodiment of the present invention. FIG. 10 shows a schematic block diagram of a bio-cell detection apparatus according to a sixth embodiment of the present invention.

In order to improve the sensitivity of measurement, the bio-cell detection apparatus 40 of the third embodiment can include, for example but not limited to, a multiplexer 27 within the package of the chip or on a printed circuit board (as shown in FIG. 7), to multiplex control the reference bio-cell detection chip 51 and plural bio-cell detection chips 41. The bio-cell detection chips 41 can be arranged in, for example but not limited to, a 1×N array (as shown in FIG. 7). The multiplexer 27 selectively decides one of the reference bio-cell detection chip 51 and plural bio-cell detection chips 41 to output its data.

Or, as shown in FIG. 8, the bio-cell detection apparatus 50 of the fourth embodiment can include, for example but not limited to, at least two multiplexers 28 and 29, to multiplex control the reference bio-cell detection chip 51 and plural bio-cell detection chips 41. The bio-cell detection chips 41 can be arranged in, for example but not limited to, a N×N array (as shown in FIG. 8). The multiplexers 28 and 29 selectively decide one of the reference bio-cell detection chip 51 and plural bio-cell detection chips 41 to output its data.

In the bio-cell detection apparatus 60 shown in FIG. 9, there can be plural bio-cell detection chip 41 which can be arranged, for example but not limited to, in serial. Or, in the bio-cell detection apparatus 70 shown in FIG. 10, there can be plural bio-cell detection chip 41 which can be arranged, for example but not limited to, in parallel.

The bio-cell detection apparatuses 40, 50, 60 and 70 shown in FIGS. 7˜10, all possess the same advantages and efficacies as the bio-cell detection apparatuses 20 and 30; moreover, they can obtain plural data at one time.

In comparison to the prior art, the present invention has advantages including: (1) The present invention can perform a broad range of detections toward different targets to be tested. (2) The steps of the present invention are simpler. (3) During the detection process, the cleaning step (to wash off the reagent) will not wash away the targets to be tested to cause an error. (4) The present invention can detect plural data at one time.

The present invention has been described in considerable detail with reference to certain preferred embodiments thereof. It should be understood that the description is for illustrative purpose, not for limiting the scope of the present invention. An embodiment or a claim of the present invention does not need to achieve all the objectives or advantages of the present invention. The title and abstract are provided for assisting searches but not for limiting the scope of the present invention. Those skilled in this art can readily conceive variations and modifications within the spirit of the present invention. For example, a device which does not substantially influence the primary function of a signal can be inserted between any two devices in the shown embodiments, such as a switch or a resistor. For another example, to perform an action “according to” a certain signal as described in the context of the present invention is not limited to performing an action strictly according to the signal itself, but can be performing an action according to a converted form or a scaled-up or down form of the signal, i.e., the signal can be processed by a voltage-to-current conversion, a current-to-voltage conversion, and/or a ratio conversion, etc. before an action is performed. It is not limited for each of the embodiments described herein before to be used alone; under the spirit of the present invention, two or more of the embodiments described hereinbefore can be used in combination. For example, two or more of the embodiments can be used together, or, a part of one embodiment can be used to replace a corresponding part of another embodiment. In view of the foregoing, the spirit of the present invention should cover all such and other modifications and variations, which should be interpreted to fall within the scope of the following claims and their equivalents.

What is claimed is:

1. A bio detection method, comprising the steps of:
(A) pre-calibrating an initial value of a bio-cell detection chip in absence of a biological reagent in the bio-cell detection chip;
(B) introducing the biological reagent into the bio-cell detection chip in absence of a sample to be tested in the bio-cell detection chip, and measuring a first electrical parameter between a pair of opposing electrodes of the bio-cell detection chip;
(C) introducing the biological reagent and the sample to be tested into the bio-cell detection chip, and measuring a second electrical parameter between the pair of opposing electrodes of the bio-cell detection chip; and
(D) comparing the second electrical parameter of the step (C) and the first electrical parameter of the step (B), to determine whether or not a target biomolecule to be detected is present.

2. The bio detection method of claim 1, wherein the step (A) includes:
(A1) outputting the initial value of the bio-cell detection chip;
(A2) determining whether the initial value falls within an acceptable range;
(A3) when the initial value falls within the acceptable range, proceeding to the step (B); and
(A4) when the initial value does not fall within the acceptable range, calibrating the initial value so that the initial value falls within the acceptable range.

3. The bio detection method of claim 2, wherein the step (A) further includes:
inputting an excitation signal to the bio-cell detection chip.

4. The bio detection method of claim 1, wherein the step (B) includes:
(B1) outputting a first output signal which is related to the first electrical parameter;
(B2) determining whether the first output signal reaches a basic level;
(B3) when the first output signal reaches the basic level, proceeding to the step (C); and
(B4) when the first output signal does not reach the basic level, adjusting a level of the first output signal to the basic level.

5. The bio detection method of claim 4, wherein the step (B) further includes: inputting an excitation signal to the bio-cell detection chip.

6. The bio detection method of claim 1, wherein the step (C) includes:
(C1) outputting a second output signal which is related to the second electrical parameter.

7. The bio detection method of claim 6, wherein the step (C) further includes:
inputting an excitation signal to the bio-cell detection chip.

8. The bio detection method of claim 1, wherein the biological reagent includes a buffer solution or an electrolyte solution.

9. The bio detection method of claim 1, wherein the first electrical parameter includes an impedance value or a capacitance value and the second electrical parameter includes an impedance value or a capacitance value.

10. A bio-cell detection apparatus, comprising:
at least one bio-cell detection chip, configured to operably carry a biological reagent and/or a sample to be tested;
a detection circuit, configured to operably output an initial value and/or a measure signal of the bio-cell detection chip, wherein the measure signal includes:
a first electrical parameter measured between a pair of opposing electrodes of the bio-cell detection chip when the biological reagent is introduced into the bio-cell detection chip but the sample to be tested is not introduced into the bio-cell detection chip; and/or
a second electrical parameter measured between the pair of opposing electrodes of the bio-cell detection chip when both the biological reagent and the sample to be tested are introduced into the bio-cell detection chip at the same time;
a control circuit, configured to operably determine whether the initial value falls within an acceptable range, or determine whether the first electrical parameter reaches a basic level;
a calibration circuit, configured to operably pre-calibrate the initial value within the acceptable range according to a determination result outputted from the control circuit when the biological reagent is not introduced into the bio-cell detection chip; and
an adjustment circuit, configured to operably adjust a level of the first output signal to the basic level according to the determination result outputted from the control circuit.

11. The bio-cell detection apparatus of claim 10, further includes:
a multiplexer, which is configured to operably multiplex control a plurality of bio-cell detection chips, wherein the plurality of bio-cell detection chips are arranged in a 1×N array.

12. The bio-cell detection apparatus of claim 10, further includes:
at least two multiplexers, which are configured to operably multiplex control a plurality of bio-cell detection chips, wherein the plurality of bio-cell detection chips are arranged in a N×N array.

13. The bio-cell detection apparatus of claim 10, wherein there are plural bio-cell detection chips and the plural bio-cell detection chips are arranged in serial.

14. The bio-cell detection apparatus of claim 10, wherein there are plural bio-cell detection chips and the plural bio-cell detection chips are arranged in parallel.

15. The bio-cell detection apparatus of claim 10, wherein the biological reagent includes a buffer solution or an electrolyte solution.

16. The bio-cell detection apparatus of claim 10, wherein the first electrical parameter includes an impedance value or a capacitance value and the second electrical parameter respectively includes an impedance value or a capacitance value.

17. A bio detection method, comprising the steps of:
(A) pre-calibrating a first initial value of a reference bio-cell detection chip in absence of a biological reagent in the reference bio-cell detection chip;
(B) introducing the biological reagent into the reference bio-cell detection chip in absence of a sample to be tested in the reference bio-cell detection chip, and measuring a first electrical parameter between a pair of opposing electrodes of the reference bio-cell detection chip;
(C) pre-calibrating a second initial value of a test bio-cell detection chip in absence of the biological reagent in the test bio-cell detection chip;
(D) introducing the biological reagent and the sample to be tested into the bio-cell detection chip, and measuring a second electrical parameter between a pair of opposing electrodes of the test bio-cell detection chip; and
(E) comparing the second electrical parameter of the step (D) and the first electrical parameter of the step (B), to determine whether or not a target biomolecule to be detected is present.

18. The bio detection method of claim 17, wherein the biological reagent includes a buffer solution or an electrolyte solution.

19. The bio detection method of claim 17, wherein the first electrical parameter includes an impedance value or a capacitance value and the second electrical parameter respectively includes an impedance value or a capacitance value.

20. A bio-cell detection apparatus, comprising:
a reference bio-cell detection chip, configured to operably carry a biological reagent;
at least one test bio-cell detection chip, configured to operably carry the biological reagent and/or a sample to be tested;
a detection circuit, configured to operably output a first initial value of the reference bio-cell detection chip and/or a second initial value of the test bio-cell detection chip, and configured to operably compare a first measure signal of the reference bio-cell detection chip and a second measure signal of the test bio-cell detection chip;
wherein the first measure signal includes:
a first electrical parameter, measured between a pair of opposing electrodes of the reference bio-cell detection chip when the biological reagent is introduced into the reference bio-cell detection chip but the sample to be tested is not introduced into the reference bio-cell detection chip;
wherein the second measure signal includes:
a second electrical parameter measured between a pair of opposing electrodes of the test bio-cell detection chip when the biological reagent and the sample to be tested are introduced into the test bio-cell detection chip;
a control circuit, configured to operably determine whether the first initial value of the reference bio-cell detection chip or the second initial value of the test bio-cell detection chip falls within an acceptable range and/or determine whether the first electrical parameter reaches a basic level;
a calibration circuit, configured to operably pre-calibrate the first initial value within the acceptable range according to a determination result outputted from the control circuit when the biological reagent is not introduced into the reference bio-cell detection chip, and/or to operably pre-calibrate the second initial value within the acceptable range according to the determination result outputted from the control circuit when the biological reagent is not introduced into the test bio-cell detection chip; and
an adjustment circuit, configured to operably adjust a level of the first output signal to the basic level according to the determination result outputted from the control circuit.

21. The bio-cell detection apparatus of claim 20, further including:
a multiplexer, which is configured to operably multiplex control the reference bio-cell detection chip and a plurality of test bio-cell detection chips, wherein the reference bio-cell detection chip and the plurality of test bio-cell detection chips are arranged in a 1×N array.

22. The bio-cell detection apparatus of claim 20, further including:
at least two multiplexers, which are configured to operably multiplex control the reference bio-cell detection chip and a plurality of test bio-cell detection chips, wherein the reference bio-cell detection chip and the plurality of test bio-cell detection chips are arranged in a N×N array.
